(19) United States
(12) Patent Application Publication
(10) Pub. No.: US 2024/0345023 A1
(43) Pub. Date: Oct. 17, 2024

(54) AQUATIC TOTAL ALKALINITY MEASUREMENT SYSTEM AND METHOD

(71) Applicant: PCFR SAS, Cucuron (FR)

(72) Inventors: Lane Hoy, Vaugines (FR); Xavier Darok, Verquieres (FR); Sebastien Ettling Coeffier, Cabrières d'Aigues (FR); Rene Brunier, Vaugines (FR)

(21) Appl. No.: 18/300,030

(22) Filed: Apr. 13, 2023

(51) Int. Cl. G01N 27/416; G01N 27/27; G01N 27/36; G01N 33/18

(52) U.S. Cl. CPC G01N 27/4165; G01N 27/27; G01N 27/36; G01N 27/4167; G01N 27/4168; G01N 33/18

(57) ABSTRACT

The aquatic total alkalinity measurement system (100) comprises:
a pH probe (105) configured to measure pH at the boundary layer of a body of water,
a probe controller (115), configured to sequentially activate and deactivate, or connect and disconnect, the pH probe,
a pH measurement variation detection device (120), configured to detect a variation of pH measurement in a sequence of pH probe measurements, and
an aquatic total alkalinity value determination device (125), configured to determine an aquatic total alkalinity value of the body of water as a function of the pH measurement variation detected, and
preferably a total alkalinity regulation unit, configured to increase or decrease the total alkalinity of the body of water as a function of the measured total alkalinity value and a target total alkalinity value.

### TECHNICAL FIELD OF THE INVENTION

The present invention relates to an aquatic total alkalinity measurement system and an aquatic total alkalinity measurement method. It applies, in particular, to the field of water treatment and to the field of in situ water total alkalinity measurement. The present invention applies to recreational aquatics (pools, spas, spray pads, water features, water fountains, water parks, wellness facilities, therapy facilities, lazy rivers, etc.) and to any similar industry or market segment where water is treated and/or monitored in a semi closed and/or closed circuit (such as waste water, water reuse, industrial water, drinking water, animal water, etc.) and to any similar industry or market segment where water is used as part of the process (such as evaporative cooling for energy generation and data centers, heating, ventilation and air conditioning, fire suppression stored water management, etc.).

### BACKGROUND OF THE INVENTION

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Total alkalinity is a key parameter of a body of water which significantly influences the capacity of chemical treatment to obtain a nominal state for that body of water.

Total alkalinity in water refers to the measure of the water's ability to neutralize acids. It is the total amount of alkaline substances (such as carbonates, bicarbonates, and hydroxides) present in the water and is expressed in milligrams per liter (mg/L) or parts per million (ppm). Maintaining proper total alkalinity levels in the water is crucial for ensuring the stability of pH (or “potential of hydrogen”) levels and preventing risk to users (such users could be bathers in a swimming pool with uncomfortable water creating skin irritation and/or health issues), damage to equipment, and/or damage to the containing structure itself.

pH has a significant influence on disinfection efficiency with many common disinfectants used or allowed in aquatics. When unstable increasing pH will dramatically and rapidly reduce disinfection efficiency, putting swimmers and facilities at risk.

Decreasing total alkalinity decreases pH stability, and increasing total alkalinity increases pH stability. A minimum total alkalinity level of 80 mg/l is generally considered to be a minimum for stable pH and predictable pH control and thus also disinfection regulation.

While total alkalinity can be measured with industrial equipment (typically by titration, or with the use of reagents) or even simple test strips, affordable simply real-time measurement does not exist because adding sensors and circulation control equipment adds complexity and cost generally deemed unsuitable in this application.

Currently, measurement of alkalinity is performed at a pH of 4.3, where bicarbonate (HCO₃⁻) turns into carbonic acid (H₂CO₃). Total alkalinity measures the amount of acid (H₃O⁺) needed to neutralize the bicarbonate (HCO₃⁻) in addition to carbonate (CO₃⁻) and hydroxide (OH) in 100 ml of water. Currently, such systems require the use of reagents that are consumables. In current systems, total alkalinity measurement in water treatment is typically performed on site, using mono- or multi-parameter sensing devices (test strips or photo-colorimetric devices). Such systems require notable processing time, the use of expert and experienced labor, and dedicated tools and consumables.

### SUMMARY OF THE INVENTION

The present invention aims at overcoming the above-mentioned drawbacks as well as other drawbacks that could be overcome although not mentioned in the description below.

The inventors have discovered that a discontinuous measurement of pH, in a body of water, using a probe and a floating reference (sometimes called “solution earth” or “liquid junction”) results in a variation of the pH measured, and that the quantification of this variation is tied to the total alkalinity value of the body of water. Hence, the present invention allows for the measurement of the total alkalinity value of a body of water without using an independent, complex, and costly total alkalinity measurement device or technique.

Such an invention does not require the use of reagents or consumables. Furthermore, such an invention does not require the water to be at a specific pH.

The present invention provides a practical and simple way of measuring total alkalinity in water treatment using a specific probe in contact with water. This in situ data measurement method can be combined with cloud-based data management and algorithms (including the use of artificial intelligence and/or machine learning) to create predictive metrics representative of the evolution of water parameters.

The in situ measurement probe can be placed in an analysis chamber, or in a pipe where water flows, or in a device inside the water. This allows a real time and in situ measurement without need for human intervention. The use of cloud-based data management allows users to have access to the total alkalinity values and behavior without being physically on site. All the data can be recorded to create a measurement log, and based on predictive algorithm, alerts may be sent to users with suitable recommended actions to adjusts total alkalinity and water balance depending on the predicted risks level. Alternatively, regulation of total alkalinity can be achieved with algorithmic control of suitable feeder or dosing equipment.

Using such an invention, the following benefits can be achieved:
reliable electronic measurement of water parameters which are traditionally negatively affected by commonly occurring electrical currents and charge in water; these currents are always present due water/pipe friction and water turbulence cause by connectors, elbows, pump, filter,
in particular embodiments, for more accurate, stable and reliable pH measurement, the use of floating reference technology (sometimes called “solution earth” or “liquid junction”) could be optionally added and thus ensures that the probe and measurement electronics equipotential with the water being sampled, and thus eliminating the impact of any electrical charge and the result allow having smaller variations in measured pH to be used to determine total alkalinity,
measuring pH periodically (rather than constantly) allows for the detection of a correlation between pH change and total alkalinity:
   repeated periodic electronic measurement of pH in the same water causes change in measured pH,
the amplitude of this measured pH change is proportional to the total alkalinity in the water sample, and
the amplitude of pH change over multiple pH measurements can be used to deduce and derive a measure of total alkalinity quantitatively.
measuring pH periodically (rather than constantly) allows for the extension of the probe lifetime and for the reduced requirement for calibration action and thus lower maintenance and/or use of less skilled technicians.

### BRIEF DESCRIPTION OF THE DRAWINGS

Other advantages, purposes and particular characteristics of the invention shall be apparent from the following non-exhaustive description of at least one particular system and method object of this invention, in relation to the drawings annexed hereto, in which:

FIG. 1 represents, schematically, a first view of a particular embodiment of a system object of the present invention,

FIG. 2 represents, schematically, a second view of a particular embodiment of a system object of the present invention,

FIG. 3 represents, schematically, the impact of a high total alkalinity on the measured pH of a particular embodiment of a system object of the present invention,

FIG. 4 represents, schematically, the impact of a low total alkalinity on the measured pH of a particular embodiment of a system object of the present invention,

FIG. 5 represents, schematically and in the form of a flowchart, a first particular succession of steps of a method object of the present invention,

FIG. 6 represents, schematically and in the form of a flowchart, a second particular succession of steps of a method object of the present invention,

FIG. 7 represents, schematically, a third view of a particular embodiment of a system object of the present invention, and

FIG. 8 represents, schematically, a graph representing a succession of pH measurements at the boundary layer of a body of water for different total alkalinity values.

### DETAILED DESCRIPTION OF THE INVENTION

This description is not exhaustive, as each feature of one embodiment may be combined with any other feature of any other embodiment in an advantageous manner.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

According to at least one embodiment, the techniques described herein are implemented by at least one computing device. The techniques may be implemented in whole or in part using a combination of at least one server computer and/or other computing devices that are coupled using a network, such as a packet data network. The computing devices may be hard-wired to perform the techniques or may include digital electronic devices such as at least one application-specific integrated circuit (ASIC) or field programmable gate array (FPGA) that is persistently programmed to perform the techniques or may include at least one general purpose hardware processor programmed to perform the techniques pursuant to program instructions in firmware, memory, other storage, or a combination. Such computing devices may also combine custom hard-wired logic, ASICs, or FPGAs with custom programming to accomplish the described techniques. The computing devices may be server computers, workstations, personal computers, portable computer systems, handheld devices, mobile computing devices, wearable devices, body mounted or implantable devices, smartphones, smart appliances, internetworking devices, autonomous or semi-autonomous devices such as robots or unmanned ground or aerial vehicles, any other electronic device that incorporates hard-wired and/or program logic to implement the described techniques, one or more virtual computing machines or instances in a data center, and/or a network of server computers and/or personal computers.

According to at least one embodiment, the present invention makes use of software, stored as instructions in a memory, ROM or storage that may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor. While each processor or core of the processor executes a single task at a time, computer system may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

It should be noted that the figures are not to scale.

It should be noted that, below, the terms “boundary layer” refer to a layer of more or less stationary fluid (such as water or air) immediately surrounding an immersed object in relative motion with the fluid.

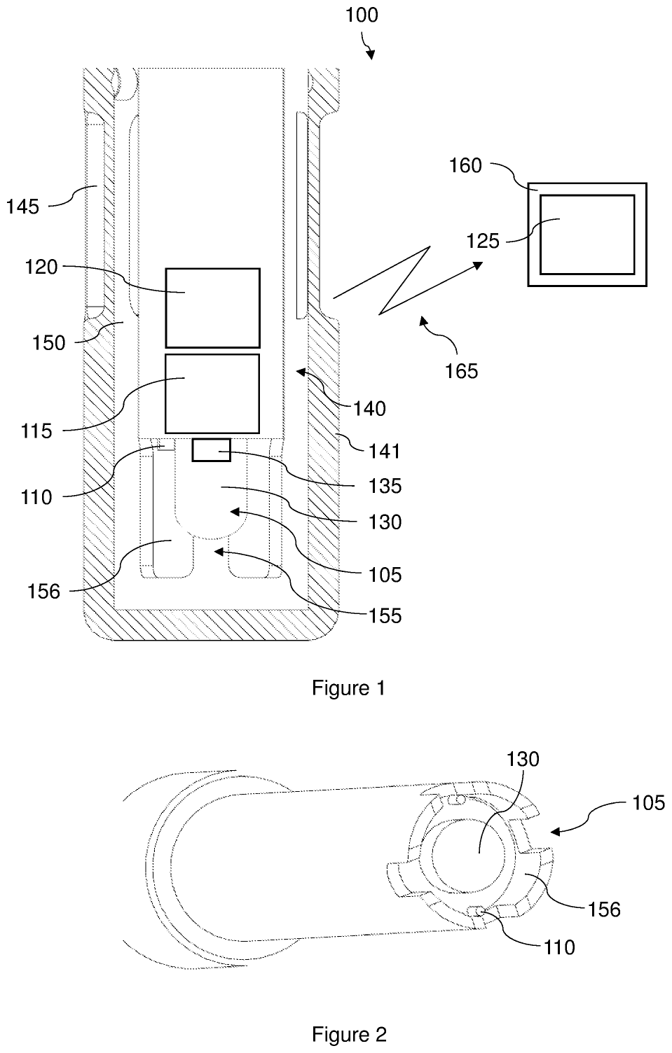

FIG. 1 represents, schematically, a particular embodiment of the system 100 object of the present invention. This aquatic total alkalinity measurement system 100, comprises:
a pH probe 105 configured to measure pH at the boundary layer of a body of water,
optionally, a floating reference device 110 in proximity of the pH probe,
a probe controller 115, configured to sequentially activate and deactivate the pH probe,
a pH measurement variation detection device 120, configured to detect a variation of pH measurement in a sequence of pH probe measurements, and
an aquatic total alkalinity value determination device 125, configured to determine an aquatic total alkalinity value of the body of water as a function of the pH measurement variation detected.

The pH probe 105 can be of any type known to a person skilled in the art that is suited for the particular implementation and intended use of the system 100. Such a pH probe 105 may differ in nature depending on the context of use of the system 100. For example, in a swimming pool, the pH probe 105 may comprise an oxidation-reduction potential sensor 135.

The objective of the pH probe 105 is to allow for the reproductible measurement of the pH in a body of water. Such a pH probe 105 is typically electronic and requires the supply of electrical energy to function. Such a pH probe 105 may further comprise a digital switch, allowing for the selective activation/deactivation of at least part of the core components of the pH probe 105.

The pH probe 105 may be mechanically located at the distal end of a sensor body, such as shown in FIGS. 1 to 4. The purpose of such a sensor body is the insertion in the body of water and, in preferred embodiments, within an analysis chamber 140.

The reference floating device 110, sometimes called “solution earth” or “liquid junction”, can correspond to any electrically conductive electrode or pin configured to normalize the signal sensed by the pH probe 105, avoiding electric noise in the proximity of the pH probe 105.

In the example shown in FIGS. 1 to 4, the reference floating device 110 comprises two electrodes, each located on a different side of a sensor 135 of the probe 105. Such electrodes may be diametrically opposed, with the sensor 135 acting as the center of a circle in which both electrodes are located on the periphery of said circle, for example. Such electrodes and the sensor 135 may be geometrically aligned.

In particular embodiments, such as the one shown in FIG. 1, the pH probe 105 comprises:
the floating reference device 110,
a microporous glass bulb membrane 130, and
an oxidation-reduction potential sensor 135.

pH measurement is based on the relationship between the concentration of H⁺ ions in tested water and the difference in electrochemical potential which is established in the lead-free glass bulb membrane of the probe. This lead-free bulb membrane is specifically designed to be selective to H⁺ ions concentration.

In general, the pH probe 105 is made of a simple electronic amplifier and a combined electrode, which consists of two electrodes: one whose potential is known and constant and the other whose potential varies with the pH.

Once the probe 105 is in contact with water, the H⁺ ions exchange on the glass bulb, creating an electrochemical potential across the bulb. The electronic amplifier detects the difference in electrical potential between the two electrodes generated in the measurement and converts the potential difference to pH units.

The pH value is determined by correlation because the potential difference between the two electrodes evolves proportionally to the pH according to the Nernst equation.

The probe controller 115 is, for example, an electronic circuit configured to electrically or electronically activate and deactivate, or connect and disconnect, the pH probe 105 or the sensor of said pH probe 105. Such an activation/deactivation or connection/disconnection may be performed by cutting and restoring power supply to the pH probe 105 or sensor or by emitting an activation/deactivation or connection/disconnection command to said pH probe 105 or sensor or relay.

The terms “activate and deactivate” relate to any hardware or software level activation/deactivation and/or to the connection/disconnection of the pH probe 105.

The probe controller 115 may itself be activated as a function of a command emitted by a computing device, located on site and mechanically connected to the pH probe 105 and/or the probe controller 115 or remotely located and connected to the probe controller 115 by way of a data connection.

The probe controller 115 may comprise, for example, a computer software executed upon a computing device, said computer software triggering the activation/deactivation or connection/disconnection of the pH probe 105. Such a computer software may correspond, for example, to a particular firmware or driver. Such a computer software may be updated remotely, and such an update may be automatically installed in the system 100.

The probe controller 115 may be configured to activate or connect the pH probe 105 periodically. The pH probe may be activated or physically connected, for example, every 60 seconds. Such an activation or physical connection may be conditional, for example to the activation of a water displacement pump. The rate of measurement may be variable depending on a mode configured. The duration of measurement may depend on the water's stability, so the pH measurement last until the measured pH is sufficiently stable.

Such an activation/deactivation can be performed by an electronic relay.

The pH measurement variation detection device 120 is, for example, an electronic device associated with the pH probe 105, configured to record a succession of pH values measured by the pH probe 105 and to compute, from said succession, a measurement variation value. Such a measurement variation value may be computed by the subtraction of a recent value from an older value.

The measured variation may be performed on immediately subsequent measured pH values or be sampled according to a particular sampling rule. Such variation may also be performed on an aggregate values of measured pH values.

For example, the pH measurement variation detection device 120 may be configured to subtract the average measured pH value during a specific, more recent timeframe from the average measured pH value during a specific, older timeframe.

For example, the pH measurement variation detection device 120 may be configured to compute determine a mathematical function fitting a succession of data points linking measured pH to time of measurement since an initial measurement. Such an example is shown in FIG. 8. In other examples, the pH measurement variation detection device 120 may be configured to store, in a memory, a succession of data points linking measured pH to time of measurement since an initial measurement.

The system 100 may further comprise a timestamping means, configured to associate a time of measurement to a sensed pH value by the pH probe 105.

The action of repeatedly measuring the pH in the same water sample induces variations in the measurement of pH, the magnitude of these variations being dependent on the total alkalinity of the water. Such a pH measurement variation detection device 120 may also correspond to a computer software executed upon a computing device.

The pH measurement variation detection device 120 may operate remotely from the pH probe 105. In such a case, the system 100 may further comprise a communication means 165 to transmit data from the pH probe 105 to the pH measurement variation detection device 120. In such a case, the pH measurement variation detection device 120 may correspond to a computer program executed by a computing server, accessible on the cloud, via a data network such as the Internet for example.

The aquatic total alkalinity value determination device 125 is, for example, an electronic device associated with the pH measurement variation detection device 120, configured to associate a total alkalinity value to the measured variation.

For example, the total alkalinity value determination device 125 may be configured to compute the derivative of a mathematical function fitting a succession of data points linking measured pH to time of measurement since an initial measurement. Such an example is shown in FIG. 8.

The total alkalinity value determination device 125 may be configured to associate, with specific or ranges of said derivatives, a specific or a range of total alkalinity value.

For example, in FIG. 8:
a first series 805 of pH measurements (Y-axis), at specific times (X-axis), measured in minutes, since an initial measurement, for a total alkalinity value of 220 mg/l,
a second series 810 of pH measurements (Y-axis), at specific times (X-axis), measured in minutes, since an initial measurement, for a total alkalinity value of 125 mg/l, and
a third series 815 of pH measurements (Y-axis), at specific times (X-axis), measured in minutes, since an initial measurement, for a total alkalinity value of 19 mg/l.

Obtaining such series linking pH to alkalinity value relationship can be performed by empirically measuring, for different values of total alkalinity and a determined activation/connection frequency for the pH sensor, values of pH in the boundary layer of a body of water and storing these series in a memory. The number of such tests to be performed is limited in terms of scope, considering the limited number of values for alkalinity.

Such a total alkalinity value may be a mathematical function of the measured variation. Such a mathematical function may be performed by determining a regression function based upon the pH series captured, or derivative values of these series, as well as the operational parameters associated with the capture.

Such derivative values may be, for example, any type of averages or parameters of derivative functions.

For example, the following mathematical formula may be used (with initial parameters values: pH=7.4; water temperature=20° C.; ORP=700 mV; flow rate=0 m³/h):

$$Alk = - 0.0001 ( {AVG}_{1} - {AVG}_{2} ) + 0.1468$$

Where:
Alk designates the total alkalinity value,
AVG₁ designates the average pH values measured from 20 seconds to 80 seconds after the initial measurement, and
AVG₂ designates the average pH values measured from 300 seconds to 360 seconds after the initial measurement.
Such a function may be approximated to Alk=(AVG₁−AVG₂).

From such a function, and initial parameters, the following correspondence table may be obtained:

| Total alkalinity value | AVG₁ − AVG₂ |
|---|---|
| 10 | 0.1368 |
| 20 | 0.1268 |
| 30 | 0.1168 |
| 40 | 0.1068 |
| 50 | 0.0968 |
| 60 | 0.0868 |
| 70 | 0.0768 |
| 80 | 0.0668 |
| 90 | 0.0568 |
| 100 | 0.0468 |
| 110 | 0.0368 |
| 120 | 0.0268 |
| 130 | 0.0168 |
| 140 | 0.0068 |
| 150 | −0.0032 |
| 160 | −0.0132 |
| 170 | −0.0232 |
| 180 | −0.0332 |
| 190 | −0.0432 |
| 200 | −0.0532 |

Such a total alkalinity value may be determined as a function of the measured variation and a preset threshold value, representative of a particular total alkalinity value.

Such an aquatic total alkalinity value determination device 125 may also correspond to computer software executed upon a computing device.

The aquatic total alkalinity value determination device 125 may operate remotely from the pH probe 105 and/or the pH measurement variation detection device 120. In such a case, the system 100 may further comprise a communication means 165 to transmit data from the pH measurement variation detection device 120 to the aquatic total alkalinity value determination device 125. In such a case, the aquatic total alkalinity value determination device 125 may correspond to a computer program executed by a computing server, accessible on the cloud, via a data network such as the Internet for example.

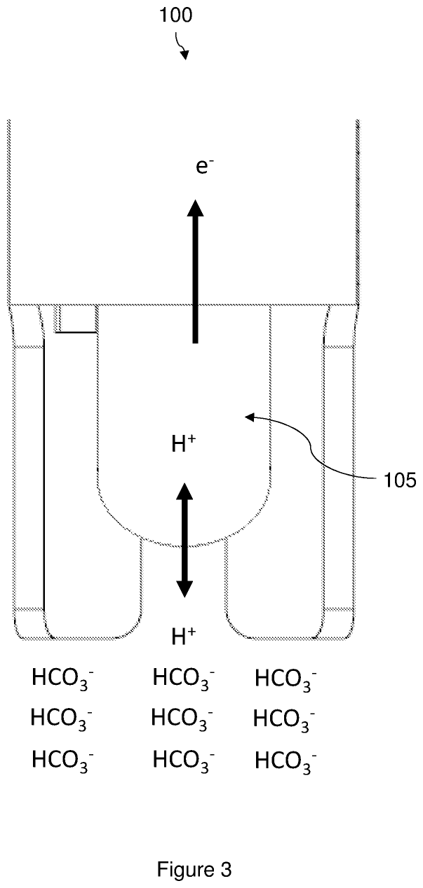

FIG. 3 shows the impact of a high total alkalinity body of water upon the system 100 object of the present invention.

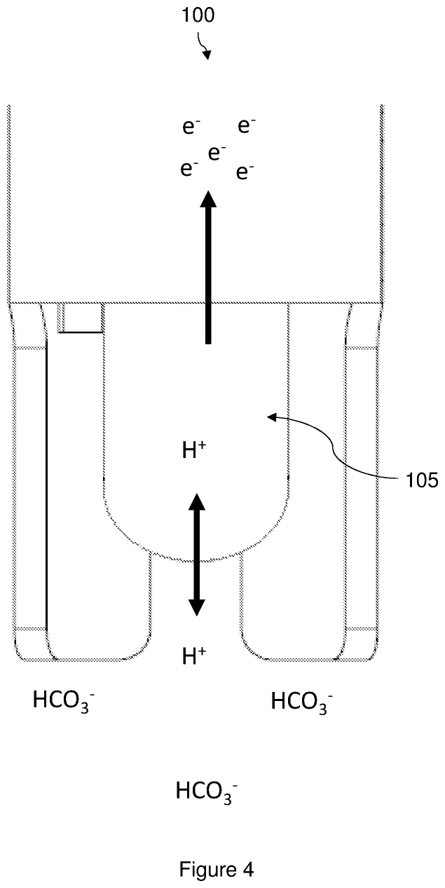

FIG. 4 shows the impact of a low total alkalinity body of water upon the system 100 object of the present invention.

In particular embodiments, the pH probe controller 115 is configured to sequentially activate and deactivate, or connect and disconnect, the pH probe 105 in a body of water with no flow. Such a state may be reached by stopping a pumping system introducing water in the body of water. In particular variants, the pH probe 105 may be activated after an absence of flow is detected (by a flow sensor, for example). In particular variants, a chamber in which the pH probe 105 is located may comprise valves that may be closed prior to the operation of the pH probe 105 activation/deactivation or connection/disconnection sequence.

The terms “body of water with no flow” designate a body of water with limited water flowing. In such a body of water, the water may circulate, but limited new water may enter.

In particular embodiments, the pH probe 105 is configured to be positioned in a small-volume body of water. Such a small volume may correspond to, for example, 1 to 2 milliliters.

The terms “small-volume body of water” designate a body of water in which the chemical reaction taking place during an interval of deactivation/activation, or connection/disconnection, of the pH probe 105 provides significant impact on the pH measure so as to show a variation between two successive measurements of the pH by the pH probe 105.

In particular embodiments, the system 100 object of the present invention comprises an analysis chamber 140, comprising an opening 145, a main volume 150 connected to the opening 145 and a recess 155 in the main volume 150, the pH probe 105 being in contact with the water in the recess 155.

The analysis chamber 140 may comprise a sensor housing 141 delimiting an internal volume in which the pH probe 105 or a sensor body associated with said pH probe 105 may be inserted.

The analysis chamber 140 is preferably configured to limit the flow of water and the volume of water in proximity to the pH probe 105. Such a configuration may be performed by selecting dimensions that limit the quantity of water entering the analysis chamber 140.

The analysis chamber 140 comprises an opening 145, of arbitrary dimensions, which allows for the passage of water from the body of water to the proximity of the pH probe 105.

The analysis chamber 140 comprises a main volume 150, defined for example by the interior dimensions of the sensor housing 141.

The analysis chamber 140 comprises a recess 155, defined by a subset of the interior dimensions of the sensor housing 141. In particular embodiments, the recess 155 is formed by crenellated sensor body extensions 156 associated to the pH probe 105, said crenellated sensor body extensions 156 limiting the movement of water in the proximity of the pH probe 105.

There are many possible configurations of the analysis chamber 140. Such configurations preferably limit the quantity of water in proximity to the pH probe 105 and/or limit the movement of water in proximity to the pH probe 105.

In particular embodiments, the system 100 object of the present invention comprises a remote computing device 160 comprising the aquatic total alkalinity value determination device 125 and a communication means 165 between the pH measurement variation detection device 120 and the aquatic total alkalinity value determination device 125.

Such a remote computing device 160 may correspond to, for example, a computing server hosted remotely and accessible through a data network, such as the Internet for example.

The communication means 165 is, for example, a communication interface coupled to bus. Communication interface provides a two-way data communication coupling to network link(s) that are directly or indirectly connected to at least one communication network, such as a network or a public or private cloud on the Internet. For example, communication interface may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network broadly represents a local area network (LAN), wide-area network (WAN), campus network, internetwork, or any combination thereof. Communication interface may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, the communication interface sends and receives electrical, electromagnetic, or optical signals over signal paths that carry digital data streams representing various types of information.

Network link typically provides electrical, electromagnetic, or optical data communication directly or through at least one network to other data devices, using, for example, satellite, cellular, Wi-Fi, or BLUETOOTH technology. For example, network link 265 may provide a connection through a network to a host computer.

Furthermore, network link may provide a connection through network or to other computing devices via internetworking devices and/or computers that are operated by an Internet Service Provider (ISP). ISP provides data communication services through a world-wide packet data communication network represented as the internet. A server computer may be coupled to the internet. Server broadly represents any computer, data center, virtual machine, or virtual computing instance with or without a hypervisor, or computer executing a containerized program system such as DOCKER or KUBERNETES. Server may represent an electronic digital service that is implemented using more than one computer or instance and that is accessed and used by transmitting web services requests, uniform resource locator (URL) strings with parameters in HTTP payloads, API calls, app services calls, or other service calls. Computer system and server may form elements of a distributed computing system that includes other computers, a processing cluster, server farm or other organization of computers that cooperate to perform tasks or execute applications or services. Server may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server may comprise a web application server that hosts a presentation layer, application layer and data storage layer such as a relational database system using structured query language (SQL) or no SQL, an object store, a graph database, a flat file system or other data storage.

The communication means 165 can send messages and receive data and instructions, including program code, through the network(s), network link and communication interface. In the Internet example, a server might transmit a requested code for an application program through Internet, ISP, local network and communication interface. The received code may be executed by processor as it is received, and/or stored in storage, or other non-volatile storage for later execution.

In particular embodiments, the aquatic total alkalinity value determination device 125 operates an algorithm and/or a trained machine learning model to associate an aquatic total alkalinity value with a variation in measured pH.

In particular embodiments, the pH probe 105 is configured to measure the pH of the body of water in a swimming pool.

In particular embodiments, the pH probe 105 is configured to measure the pH of the body of water in a pipe.

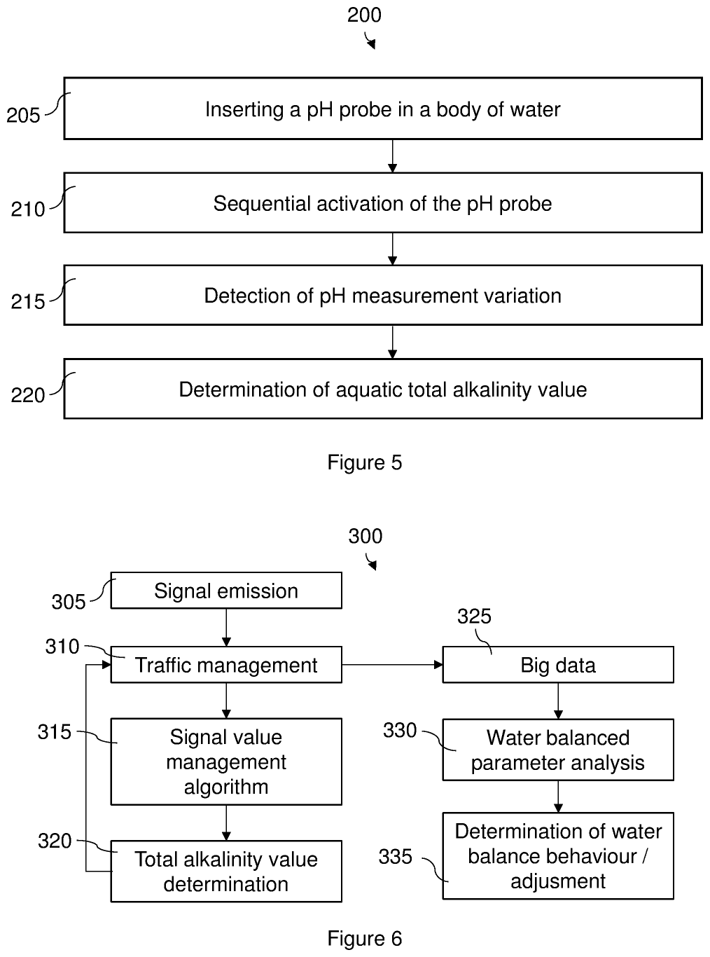

FIG. 5 shows, schematically, a particular embodiment of the method 200 object of the present invention. This aquatic total alkalinity measurement method 200 comprises:
a step 205 of inserting, in a body of water, a pH probe configured to measure pH at the boundary layer of a body of water,
a step 210 of sequential activation and deactivation, or connection and disconnection, of the pH probe,
a step 215 of detection of pH measurement variation, to detect a variation of pH measurement in a sequence of pH probe measurements, and
a step 220 of determination of an aquatic total alkalinity value, to determine an aquatic total alkalinity value of the body of water as a function of the pH measurement variation detected.

Embodiments of such a method 200 are disclosed in regard to FIGS. 1 to 3.

FIG. 6 shows, schematically, a particular embodiment of the method 300 object of the present invention. This method 300 comprises:
a step 305 of signal emission, by the pH probe 105 or the pH measurement variation detection device 120, of a measured pH or pH measurement variation,
a step 310 of traffic management, configured to address the measured pH or pH measurement variation to a specific software module and the associated hardware, and in a first stream:
   a step 315 of execution of a signal value management algorithm, configured to extract values from the measured pH or pH measurement variation, and
   a step 320 of total alkalinity value determination, as a function of the signal value management algorithm executed,
in a second stream:
   a step 325 of big data analysis of the total alkalinity value,
   a step 330 of water balanced parameter analysis, and
   a step 335 of determination of water balance behavior/adjustments to be performed upon the body of water.

As it can be understood:

The use of an integrated floating earth, a microporous glass bulb membrane and an ORP sensor, placed in an analysis chamber, allows to provide accurate, reliable and continuous data measurement.

The core of such an analysis chamber preferably fits the sensor to limit the amount of water in the chamber part dedicated to measurement. When the water is flowing, water parameters are homogeneous in this chamber part. Once the flow stops, a chemicals parameters rate gradient occurs in this chamber part that does not happen, or in a limited fashion, in the global water in the analysis chamber. This is due, in that particular example, to the ionic exchange through the microporous glass bulb membrane of the probe between the core of the probe and a reactional volume close to the probe. Other configurations for the probe would yield similar results.

The inventors have discovered the existence of pH value micro variations (considered as hysteresis) when the water is not flowing in the analysis chamber and when the pH measurement stops at the same time. Those variations are directly linked to total alkalinity value in the analysis zone.

When the total alkalinity is high, shown in FIG. 3, a high concentration of HCO₃⁻ is located in the analysis zone. These ions absorb a major part of the pH micro variation linked to the chemical reaction between H⁺ in the water and H⁺ in the probe through the microporous glass bulb membrane. These micro variations are treated by the probe and generate a low electrical signal.

When the total alkalinity is low, shown in FIG. 4, a low concentration of HCO₃⁻ is located in the analysis zone. These ions absorb a minor part of the pH micro variation linked to the chemical reaction between H⁺ in the water and H⁺ in the probe through the micro porous glass bulb membrane. These micro variations are treated by the probe and generate a high electrical signal.

These electrical signals are preferably measured thanks to the probe being associated with the floating reference device, which allows the exclusion of any background hysteresis and the signal processing to focus on exact chemicals measurement.

As shown in FIGS. 3 and 4, the intensity of the measured signal is directly linked to the total alkalinity value. The higher the total alkalinity is, the lower will be the signal, and vice-versa.

These signals can be sent and treated in a cloud-based computing solution, such as shown in FIG. 6. Once the electrical signal is emitted by the probe, it is sent by a traffic manager to a dedicated cloud application and data storage. The traffic manager can transmit the data to a specific algorithm, where the signal value is transformed into a total alkalinity value. This value can be routed to a big data management device, using the traffic manager.

Further algorithms can use the total alkalinity value to compare it to the other water balanced parameters, in order to determine the water balance behavior and needed adjustment. For example, measured total alkalinity values, can be combined with water temperature values, total hardness values, total dissolved salt values, measured pH and desired pH values, disinfection chemical ppm values, oxidation-reduction potential values, and any other values applicable to the specific application. These combinations, used with the Langelier saturation index, can give the balance behavior of the water for measured and desired pH values, and also other chemical values that depend on balanced pH conditions. Thus, it can immediately be known if the water is balanced, aggressive or scaling, and also enables estimation of the water's future behavior and condition, depending on those values, modifications and trends.

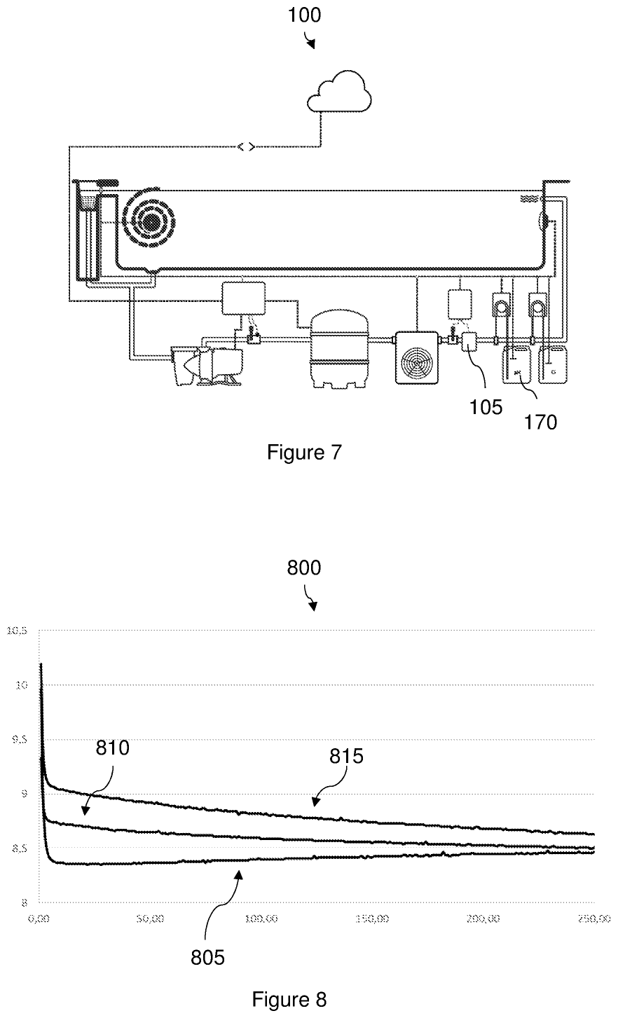

FIG. 7 represents a particular embodiment of the system 100 object of the present invention, which comprises a total alkalinity regulation unit 170, configured to increase or decrease the total alkalinity of the body of water as a function of the measured total alkalinity value and a target total alkalinity value.

The total alkalinity regulation unit 170 may correspond to any device suited for the regulation of alkalinity known to one skilled in the art. Such a device may be, for example, a mixer configured to mix acid from a reservoir and a stream of water processed in a swimming pool water treatment circuit.

The total alkalinity regulation unit 170 operates to reach a target total alkalinity in the body of water. This target total alkalinity can be set by a user or be remotely set by a computing system. Such a value may be higher or equal to 120 mg/l (or ppm) and lower than 250 mg/l (or ppm). If the measured total alkalinity is above the target total alkalinity, the regulation unit 170 may be operated to reduce the total alkalinity in the body of water whereas if the measured total alkalinity is below the target total alkalinity, the regulation unit 170 may be operated to increase the total alkalinity in the body of water.

### OBJECT OF THE INVENTION

The present invention is intended to remedy all or part of these disadvantages.

To this effect, according to a first aspect, the present invention aims at an aquatic total alkalinity measurement system, comprising:
a pH probe configured to measure pH at the boundary layer of a body of water,
a probe controller, configured to sequentially activate and deactivate, or connect and disconnect, the pH probe,
a pH measurement variation detection device, configured to detect a variation of pH measurement in a sequence of pH probe measurements, and
an aquatic total alkalinity value determination device, configured to determine an aquatic total alkalinity value of the body of water as a function of the pH measurement variation detected.

Such provisions allow for the accurate and near real-time measurement of the total alkalinity value of a body of water at an inexpensive cost and with ordinary equipment. However, the benefits of this invention result from the counter-intuitive discovery, by the inventors, that switching the pH probe on and off when the water is not flowing provides an accurate total alkalinity measurement whereas continuous pH measurement does not. This is because the successive activation/deactivation, or connection/disconnection of the pH probe results in a chemical reaction taking place in the vicinity of the pH probe. This chemical reaction results in a variation in pH measurement, said variation being dependent on the total alkalinity value of the water in the vicinity of the pH probe off, when the water is not flowing. Therefore, the present invention allows for the determination of the total alkalinity value of a body of water without using a total alkalinity measurement sensor. Such an indirect measurement significantly improves the capacity to measure total alkalinity in swimming pools and in any other aquatic installation and management systems.

In particular embodiments, the system object of the present invention comprises a floating reference device in proximity to the pH probe.

Such embodiments reduce the hysteresis of the pH probe.

In particular embodiments, the pH probe comprises:
the floating reference device,
a microporous glass bulb membrane, and
an oxidation-reduction potential sensor.

Such embodiments allow for an ionic exchange to take place in the vicinity of the microporous glass bulb membrane, said ionic exchange resulting in the measurement variations of the pH by the pH probe.

In particular embodiments, the pH probe controller is configured to sequentially activate and deactivate, or connect and disconnect, the pH probe in a non-flowing water.

The less the water in the body of water around the pH probe is renewed in between a deactivation and an activation, or the disconnection and connection, of the pH probe, the more representative of the total alkalinity of the water the variation between successive pH measurements is.

In particular embodiments, the pH probe is configured to be positioned in a low-volume body of water.

Such embodiments allow for the frequent activation/deactivation, or connection/disconnection, of the pH probe and thus more frequent measurement of total alkalinity of the body of water.

In particular embodiments, the system object of the present invention comprises an analysis chamber, comprising an opening, a main volume connected to the opening and a recess in the main volume, the pH probe being in contact with the water in the recess.

In particular embodiments, the system object of the present invention comprises a remote computing device comprising the aquatic total alkalinity value determination device and a communication means between the pH measurement variation detection device and the aquatic total alkalinity value determination device.

Such embodiments allow for the use of advanced calculation techniques in a centralized location, thus improving the quality of output of a fleet of pH probes.

In particular embodiments, the aquatic total alkalinity value determination device operates an algorithm and/or a trained machine learning model to associate an aquatic total alkalinity value with a variation in measured pH.

Such embodiments allow for the use of advanced calculation techniques in a centralized location, thus improving the quality of output of a fleet of pH probes.

In particular embodiments, the pH probe is configured to measure the pH of the body of water in a swimming pool and/or in an aquatic installation.

In particular embodiments, the pH probe is configured to measure the pH of the body of water in a pipe.

In particular embodiments, the system object of the present invention comprises a total alkalinity regulation unit, configured to increase or decrease the total alkalinity of the body of water as a function of the measured total alkalinity value and a target total alkalinity value.

According to a second aspect, the present invention aims at an aquatic total alkalinity measurement method, which comprises:
a step of inserting, in a body of water, a pH probe configured to measure pH at the boundary layer of a body of water,
a step of sequential activation and deactivation, or connection and disconnect, of the pH probe,
a step of detection of pH measurement variation, to detect a variation of pH measurement in a sequence of pH probe measurements, and
a step of determination of an aquatic total alkalinity value, to determine an aquatic total alkalinity value of the body of water as a function of the pH measurement variation detected.

The benefits of the method object of the present invention are similar to the benefits of the system object of the present invention.

1. Aquatic total alkalinity measurement system, comprising:
a pH probe configured to measure pH at the boundary layer of a body of water,
a probe controller, configured to sequentially activate and deactivate, or connect and disconnect, the pH probe,
a pH measurement variation detection device, configured to detect a variation of pH measurement in a sequence of pH probe measurements, and
an aquatic total alkalinity value determination device, configured to determine an aquatic total alkalinity value of the body of water as a function of the pH measurement variation detected.

2. System according to claim 1, which comprises a floating reference device in proximity of the pH probe.

3. System according to claim 2, in which the pH probe comprises:
the floating reference device,
a microporous glass bulb membrane, and
an oxidation-reduction potential sensor (135).

4. System according to claim 1, in which the pH probe controller is configured to sequentially activate and deactivate, or connect and disconnect, the pH probe in a stagnant body of water.

5. System according to claim 1, in which the pH probe is configured to be positioned in a low-volume body of water.

6. System according to claim 5, which comprises an analysis chamber, comprising a slotted opening, a main volume connected to the opening and a recess in the main volume, the pH probe being in contact with the water in the recess.

7. System according to claim 1, which comprises a remote computing device comprising the aquatic total alkalinity value determination device and a communication means between the pH measurement variation detection device and the aquatic total alkalinity value determination device.

8. System according to claim 1, in which the aquatic total alkalinity value determination device operates an algorithm and/or a trained machine learning model to associate an aquatic total alkalinity value with a variation in measured pH.

9. System according to claim 1, in which the pH probe is configured to measure the pH of the body of water in a swimming pool.

10. System according to claim 1, in which the pH probe is configured to measure the pH of the body of water in a pipe.

11. System according to claim 1, which comprises a total alkalinity regulation unit, configured to increase or decrease the total alkalinity of the body of water as a function of the measured total alkalinity value and a target total alkalinity value.

12. Aquatic total alkalinity measurement method, comprising:
a step of inserting, in a body of water, a pH probe configured to measure pH at the boundary layer of a body of water,
a step of sequential activation and deactivation, or connection and disconnection, of the pH probe,
a step of detection of pH measurement variation, to detect a variation of pH measurement in a sequence of pH probe measurements, and
a step of determination of an aquatic total alkalinity value, to determine an aquatic total alkalinity value of the body of water as a function of the pH measurement variation detected.
